(19) United States
(12) Patent Application Publication
(10) Pub. No.: US 2026/0272379 A1
(43) Pub. Date: Sep. 17, 2026

(54) SYSTEMS, METHODS, AND APPARATUSES FOR CONTINUOUS MONITORING AND EFFECTIVE MANAGEMENT OF HYDRATION

(71) Applicant: ARIZONA BOARD OF REGENTS ON BEHALF OF ARIZONA STATE UNIVERSITY, Scottsdale, AZ (US)

(72) Inventors: Shovito Barua Soumma, Tempe, AZ (US); Asiful Arefeen, Phoenix, AZ (US); Stavros Kavouras, Scottsdale, AZ (US); Hassan Zadeh, Phoenix, AZ (US)

(21) Appl. No.: 19/540,362

(22) Filed: Feb. 13, 2026

Related U.S. Application Data
(60) Provisional application No. 63/758,119, filed on Feb. 13, 2025.

(51) Int. Cl. A61B 5/00; A61B 5/01; A61B 5/0205

(52) U.S. Cl. CPC A61B 5/4875; A61B 5/01; A61B 5/02055; A61B 5/443; A61B 5/681; A61B 5/7267; A61B 5/7275

(57) ABSTRACT

A real time automated hydration management solution is described herein that uses predictive and counterfactual generation models to recommend optimal behavior for normalizing hydration levels based on data collected in real time.

### RELATED APPLICATIONS

This application claims the benefit of U.S. Application No. 63/758,119, filed Feb. 13, 2026.

### TECHNICAL FIELD

The disclosure herein involves systems and methods for effectively managing user hydration levels.

### BACKGROUND

Although hydration management solutions are currently available, they are either too generalized or not tailored to the needs of those with specific medical vulnerabilities. This lack of personalized, real-time hydration monitoring can lead to poor health outcomes, frequent hospital readmissions, and, in extreme cases, life-threatening situations. There is a need for a real time automated hydration management solution that recommends optimal behavior to normalize hydration levels.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A method is described herein comprising, receiving a feature vector for a plurality of subjects, wherein a feature vector comprises feature data elements of a subject, applying a predictive model to the feature vector of the plurality of subjects, wherein the predictive model classifies the plurality of subjects as hydrated or dehydrated, identifying at least one subject of the plurality of subjects that is classified as dehydrated, generating candidate counterfactual feature vectors for the at least one subject, wherein the generating the candidate counter factual feature vectors comprises identifying counterfactual changes to the feature data elements which transition the predictive model classification of the subject from dehydrated to hydrated, where the candidate counter factual feature vectors comprise only identified counterfactual feature data elements, computing a diversity distance between each possible pairing of candidate counter factual feature vectors, generating a preference aware cost for the candidate counterfactual vectors, generating an optimization score for each candidate counter factual feature vector using a weighted sum of the diversity score and the preference aware cost for each pair, applying a greedy selection process to select a subset of the counterfactual feature vectors, and providing the subset of the counterfactual feature vectors to the subject.

In embodiments, a diversity distance comprises computing a first distance between each pairing of candidate counter factual feature vectors.

In embodiments, the computing the first distance comprises calculating a first weighted L1 distance between each pair.

In embodiments, the first weighted L1 distance is calculated using first weights, wherein the first weights comprise a diversity measure assigned to each feature type of the candidate counter factual feature vectors.

In embodiments, the generating a preference aware cost comprises identifying a subset feature vector of the feature vector, wherein the subset feature vector includes feature data elements corresponding to counterfactual feature data elements of the candidate counter factual feature vectors.

In embodiments, the generating a preference aware cost comprises computing a second distance between each pairing of candidate counter factual feature vectors and the subset feature vector.

In embodiments, the second distance comprises a second weighted L1 distance between each pair.

In embodiments, the second weighted L1 distance is calculated using second weights, wherein the second weights comprise preferences of the subject to adopt behavior to achieve counterfactual changes to the feature data elements.

In embodiments, the generating candidate counterfactual feature vectors comprises applying a counterfactual generator to the counter factual feature vector of the at least one subject of the plurality of subjects.

In embodiments, the feature data elements include fluid intake.

In embodiments, the feature data elements include activity levels.

In embodiments, the feature data elements include sweat loss.

In embodiments, the feature data elements include ambient temperature.

In embodiments, the feature data elements include relative humidity.

In embodiments, the feature data elements include total body water.

In embodiments, the feature data elements include time of day.

### BRIEF DESCRIPTION OF THE DRAWINGS

FIG. 1 shows a diagram that highlights the key advantages of HydroGuard, under an embodiment.

FIG. 2 shows a counterfactual AI framework for monitoring hydration levels, under an embodiment.

FIG. 3 shows a feature space for dehydrated and hydrated subjects, under an embodiment.

FIG. 4 visually depicts a classifier space boundary provided by a neural network model, under an embodiment.

FIG. 5 illustrates generation of adversarial data points, under an embodiment.

FIG. 6 shows dismissal of original subject data in favor of adversarial points to define the classifier space boundary, under an embodiment.

FIG. 7 shows transition of a user from a dehydrated to hydrated state given change in certain behavior, under an embodiment.

FIG. 8 shows a high-level diagram of the autoencoder model, under an embodiment.

FIG. 9A shows components and specification of the classifier model, under an embodiment.

FIG. 9B shows components and specification of the autoencoder, under an embodiment FIG. 10A shows the first step: The counterfactual technique generates multiple intervention pathways, however, not all of them are feasible for the user to follow.

FIG. 10B shows the second step: Selects the most feasible intervention plan that is feasible for the user and requires minimal change/minimum number of feature change etc.

FIG. 11A shows dehydrated and hydrated data points with respect to a classifier space boundary generated by the neural net classifier, under an embodiment.

FIG. 11B shows adversarial hydrated and dehydrated data points that are generated using the autoencoder described above, under an embodiment.

FIG. 11C illustrates use of the new classifier boundary space to identify an abnormal state of a user and to provide the user with counterfactual information sufficient to transition the user from a dehydrated to a hydrated state, under an embodiment.

FIG. 12 shows data collection for purposes of training the model, under an embodiment.

FIG. 13 shows data collection for purposes of training the model, under an embodiment.

FIG. 14 shows the relationship between exposure to heat and significant physiological changes, under an embodiment.

FIG. 15 illustrates a graph representation of candidate counterfactuals for a factual instance under an embodiment.

FIG. 16 illustrates a graph of counterfactual and XT feature vectors, under an embodiment.

FIG. 17 illustrates factual-counterfactual pairs, under an embodiment.

FIG. 18 illustrates factual-counterfactual pairs under an embodiment.

FIG. 19 illustrates factual-counterfactual pairs under an embodiment.

FIG. 20 illustrates factual-counterfactual pairs under an embodiment.

### DETAILED DESCRIPTION

Hydration is critical for maintaining overall health, but managing it is complex for individuals with specific medical conditions like congestive heart failure, kidney disease, and endocrine disorders. These conditions require precise hydration management-too little fluid can lead to dehydration and serious health risks, while too much can result in fluid overload, potentially leading to life-threatening complications.

Hydration is essential but complex. Proper hydration is crucial for maintaining bodily functions, particularly for those with underlying medical conditions. High-risk groups (e.g., individuals with congestive heart failure, kidney disease, or endocrine disorders) face severe consequences from improper hydration. 70% of the human body is made up of water and is crucial for maintaining bodily functions.

Danger of fluid imbalance includes dehydration/overhydration and other serious health implications: frequent hospital readmissions, poor quality of life, increased healthcare costs.

As just one example of individual risk, Kevin is a person who, like many others, is trying to manage his health conditions while leading a normal life. Kevin has been diagnosed with Chronic Kidney Disease (CKD) and/or a heart condition, both of which require him to carefully monitor his fluid intake. Drinking too little water can lead to dehydration, making him feel weak and thirsty. On the other hand, drinking too much, especially when he's out and about in the heat, can lead to serious complications like edema, electrolyte imbalance, kidney malfunction, and even heart complications.

When Kevin is at home, he's cautious. He knows that drinking too much water could strain his kidneys and exacerbate his heart condition.

On a hot day, after some light housework, Kevin feels thirsty and weak. He takes a small sip of water, careful not to overdo it, but it leaves him wondering if it's enough.

As Kevin feels the effects of the heat, he starts drinking more water, trying to stay hydrated. However, without realizing it, he's putting himself at risk. What seems like a healthy habit—drinking water—is actually tipping the balance and could trigger severe health issues.

Kevin's dilemma is common. Many people with chronic conditions struggle to balance their hydration needs, especially when environmental factors like heat come into play. The real challenge lies in knowing when and how much to drink to stay safe as mismanagement of hydration may lead to . . .
Edema: Swelling in his legs and feet due to fluid retention.
Electrolyte Imbalance: Disrupting his body's essential salt and mineral levels.
Kidney Malfunction: Overburdening his already strained kidneys.
Heart Complications: Triggering dangerous heart conditions due to the extra fluid.

The problem of ineffective hydration arises from multiple sources including insufficient real time monitoring, lack of personalized guidance, no precise interventions and inadequate focus on vulnerable populations.

Current monitoring methods lack . . .
1. Lack of Personalized Guidance: Existing hydration recommendations are often too generic, not taking into account individual health conditions and environmental factors.
2. Insufficient Real-Time Monitoring: Most solutions do not offer real-time, actionable feedback, leaving users to guess their hydration needs. Example: A manual hydration tracker won't alert you if you need more fluids due to high heat and exercise, potentially leading to dehydration.
3. No Counterfactual Interventions: Current systems don't offer proactive advice on what to do when a user is likely to make a hydration mistake (e.g., consuming diuretics like coffee). Example: A person with a kidney condition drinks coffee, unknowingly increasing their risk of dehydration, without receiving a warning or alternative suggestion.
4. Inadequate Focus on Vulnerable Populations: Most hydration tools are designed for athletes or the general public, not for high-risk individuals who require precise and cautious fluid management. Example: Tools may encourage excessive water intake, which could be harmful for those with fluid restrictions due to heart or kidney conditions. 75% of Americans are chronically dehydrated and mild levels of dehydration can produce disruptions in mood and cognitive functioning.

Systems and method are described herein that use physiological and behavioral data about a person to continuously measure the person's hydration level and provide actionable feedback to ensure healthy and safe hydration levels.

The system uses data about a person's fluid intake, heart rate, electrodermal activity, physical activity, heart rate variability, skin temperature, ambient temperature, health condition, and other physiological and behavioral variables to estimate a person's hydration level.

The hydration level is accurately estimated using advanced AI techniques including deep neural networks. A model as further described below is optimized and compressed using compression techniques such as model pruning, model quantization, and knowledge distillation. This enables use to create models that are (although complex) lightweight and computationally simple for implementation on mobile devices, portable devices, embedded devices, and wearable sensors. The model provides a personalized estimation of the person's hydration level.

The model uses a new method based on counterfactual explanations to identify variables that should be modified in order to obtain normal and safe hydration levels.

### Prospective Intervention:

Under an embodiment, the model forecasts a person's hydration level ahead of time and provides feedback to prevent abnormal hydration levels that could have happened.

### Retrospective Intervention:

Under another embodiment, the model identifies past abnormal events (e.g., the person has been dehydrated at certain points of time in the past) and provides feedback on how the person could have prevented an abnormal event that happened in the past.

These interventions are provided in form of upward counterfactuals or downward counterfactuals, under an embodiment.

### Upward Counterfactuals

The model provides upward counterfactuals. In this case, the system provides feedback about abnormal health events (e.g., low hydration level) and how they could have been reversed by the person by taking preventative behavioral choices.

### Downward Counterfactuals

The model provides downward counterfactuals. In this case, the system provides feedback about normal health events (e.g., good hydration levels) and how the person could have experienced an abnormal health event (e.g., dehydrated) had they not taken appropriate actions (e.g., the person could have experienced dehydration if they continued exercising outside for an additional 10 minutes yesterday at 2 μm).

An approach described herein implements quality data collection, prediction of hydration levels, and provision of precise water intake required to rehydrate.

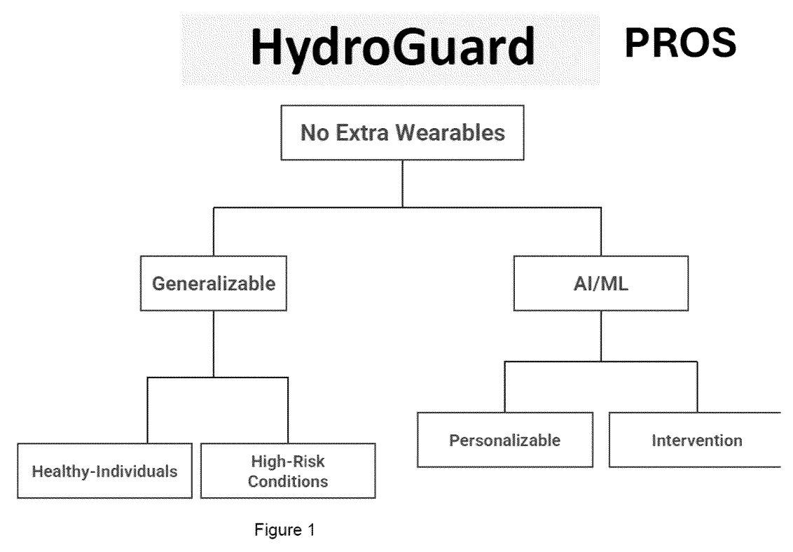

FIG. 1 shows a diagram that highlights the key advantages of HydroGuard, emphasizing its practical and innovative approach to hydration management. One of the standout features is the “No Extra Wearables” aspect, which reduces the burden on users by integrating seamlessly into their existing health-monitoring ecosystem without requiring additional devices. HydroGuard is highly generalizable, making it suitable for both healthy individuals and those with high-risk conditions, ensuring wide applicability. The system also leverages AI/ML capabilities, enabling it to provide personalized recommendations tailored to the user's unique hydration needs and lifestyle. Additionally, HydroGuard's intervention system empowers users by offering actionable suggestions, helping them prevent dehydration or overhydration proactively. Together, these features make HydroGuard an accessible, efficient, and versatile solution for hydration monitoring.

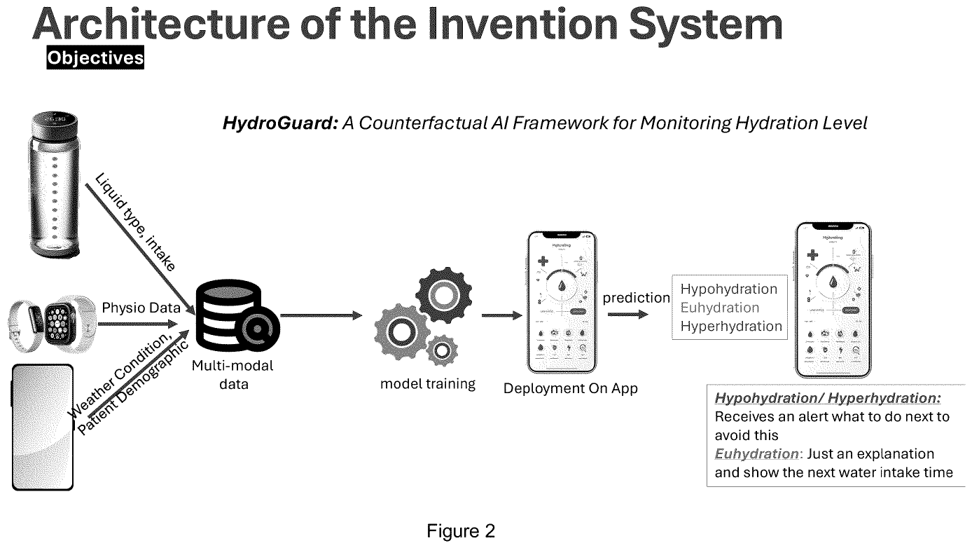

FIG. 2 shows a counterfactual AI framework for monitoring hydration level. The framework includes data collection using at least a mobile device (e.g. weather conditions, patient demographics (gender, age, BMI, weight), physiological data via wearable devices, a liquid intake using a smart water bottle, etc. The framework trains a model (described in greater detail below) using data from at least these multiple sources.

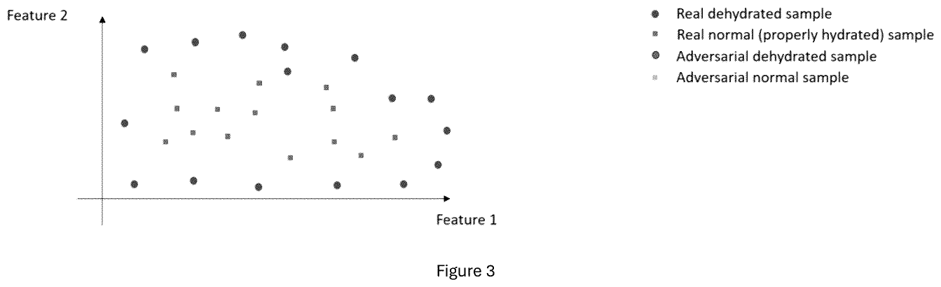

FIGS. 3-7 illustrate a decision boundary construction approach which characterizes a decision boundary with adversarial edge cases that will allow us explain user data and create followable suggestions (displayed in 2D for easy visualization).

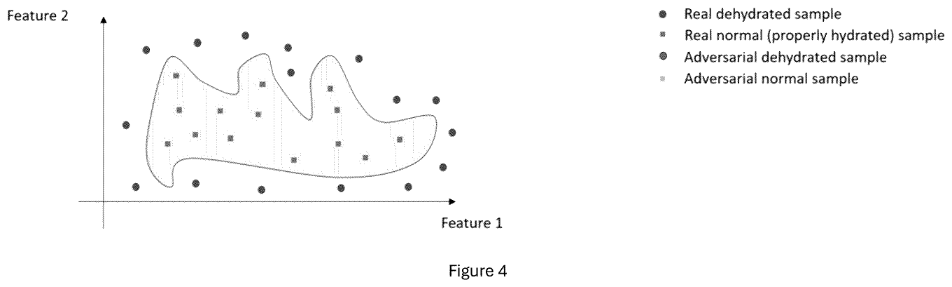

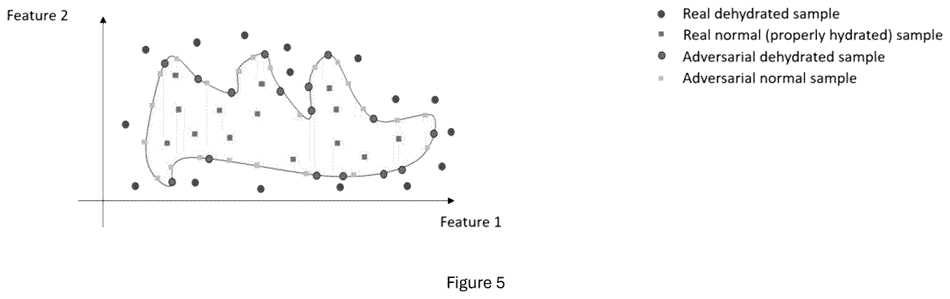

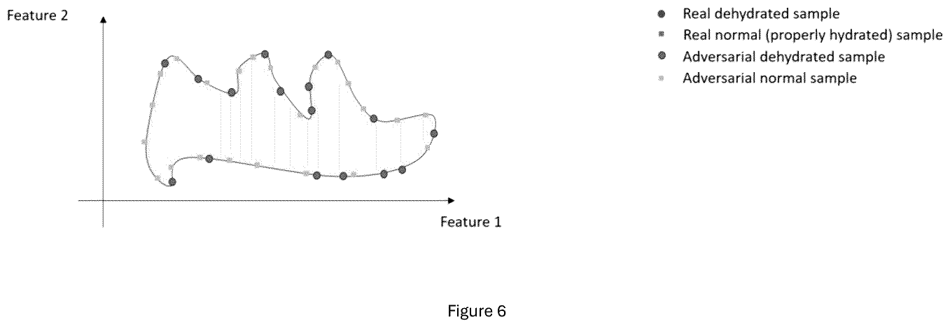

FIG. 3 shows a feature space for dehydrated and hydrated subjects (represented by red cdots and green squares). FIG. 4 visually depicts a classifier space boundary provided by a neural network model (described further below) trained on the dehydrated and hydrated subject data. FIG. 5 illustrates that once the classifier space is established, an autoencoder (as further described below) uses multi-modal physiological data (e.g., heart rate variability, electrodermal activity, skin temperature), behavioral data (e.g., fluid intake patterns, activity levels), and environmental data (e.g., ambient temperature, humidity) to generate adversarial dehydrated and hydrated samples (represented by blue dots and yellow squares). FIG. 6 shows that original subject data is discarded in favor of adversarial points that now define the classifier space boundary. Under an embodiment, the new classifier space boundary uses feature data of a user to determine a hydration state.

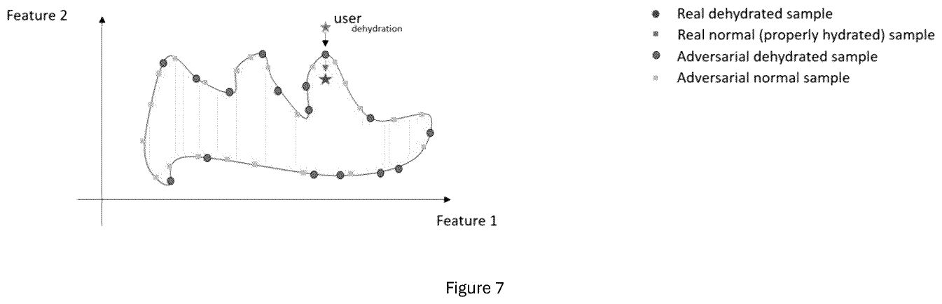

The most direct path is determined by minimizing the distance between the user's feature vector and the adversarial-defined boundary using optimization algorithms (e.g., gradient descent), which identify the smallest perturbation required to cross into the hydrated region. The system prioritizes actionable features (e.g., fluid intake, activity level) with the highest sensitivity to hydration state changes, derived from counterfactual analysis. For example, if reducing fluid intake by 200 mL moves the user's position closest to the boundary, this becomes the suggested intervention. Variable selection is guided by domain-specific constraints (e.g., medical conditions) and environmental context (e.g., heat exposure), ensuring realistic and safe behavioral adjustments (see FIG. 7).

If a user is determined to be dehydrated, then this technique may use the “feature” position of the user and the new classifier space boundary to suggest a change in behavior (e.g., a suggested level of water intake) to bring the use into a hydration state (see FIG. 7).

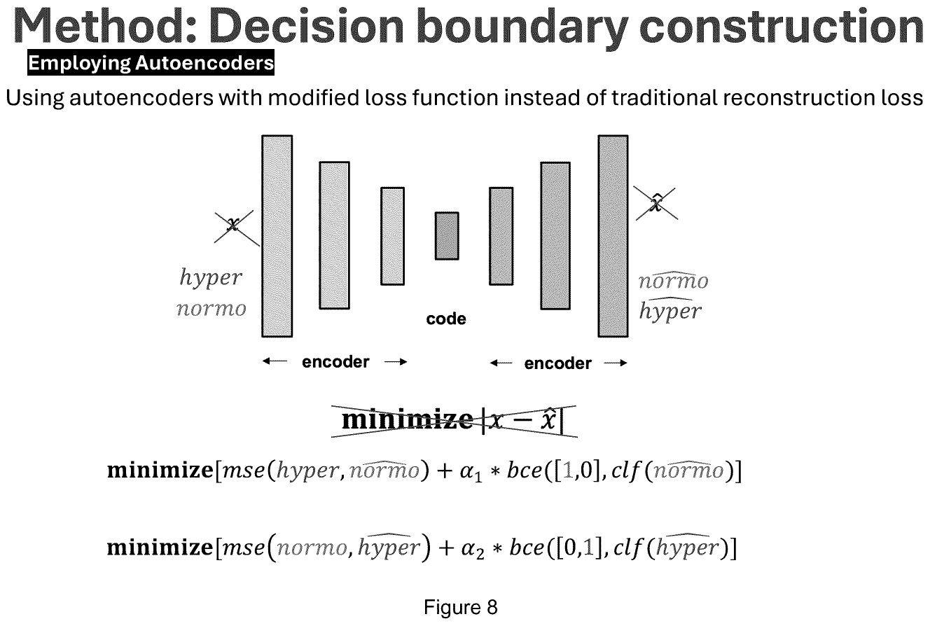

Once the classifier space boundary is established using adversarially generated dehydrated and hydrated samples, the hydration state of a user is determined by mapping real-time physiological, behavioral, and environmental feature data onto this space. This determination follows an in/out classification approach, where a user is classified as hydrated if their feature data falls within the hydrated region of the space, and dehydrated if it falls outside or near the decision boundary. However, instead of a rigid threshold-based classification, a gradient-based optimization method is employed to compute the shortest Euclidean distance from the user's current state to the nearest hydrated region within the classifier space. This allows the system to determine the most efficient transition pathway by identifying the minimum necessary changes in physiological or behavioral parameters. To achieve this, a sensitivity analysis is performed on the user's current data to rank the impact of different variables-such as heart rate variability, skin temperature, electrodermal activity, and fluid intake-on shifting the hydration state. The system then prioritizes the variable(s) that require the smallest modifications to induce a transition to the hydrated state. Mathematically, this counterfactual input generation is modeled as a constrained optimization problem, where the system minimizes the difference between the user's current feature set and a target hydrated feature set while ensuring the predicted hydration state moves to euhydration. Unlike traditional hydration monitoring systems that rely solely on threshold-based recommendations, this method leverages adversarial training to refine decision boundaries and enhance model robustness. The autoencoder framework depicted in FIG. 8 is designed with a modified loss function that incorporates multi-task learning to encode hydration state separability while leveraging adversarial training to generate synthetic edge cases for more precise classification. This approach ensures that the system does not just detect dehydration but also provides actionable and optimized hydration recommendations tailored to each user's physiological profile.

FIG. 8 shows a high-level diagram of the autoencoder model, under an embodiment. The autoencoder model uniquely integrates multimodal data inputs, including physiological signals (e.g., heart rate variability, electrodermal activity, skin temperature), behavioral data (e.g., fluid intake patterns, activity levels), and environmental factors (e.g., ambient temperature, humidity). This comprehensive integration allows the autoencoder to capture complex patterns and interactions among these diverse data types, facilitating the generation of adversarial samples that effectively define the classifier's decision boundary. The autoencoder's architecture is designed to learn a compact and informative latent representation of the input data, enabling it to produce meaningful adversarial examples that challenge and refine the classifier's ability to distinguish between hydrated and dehydrated states.

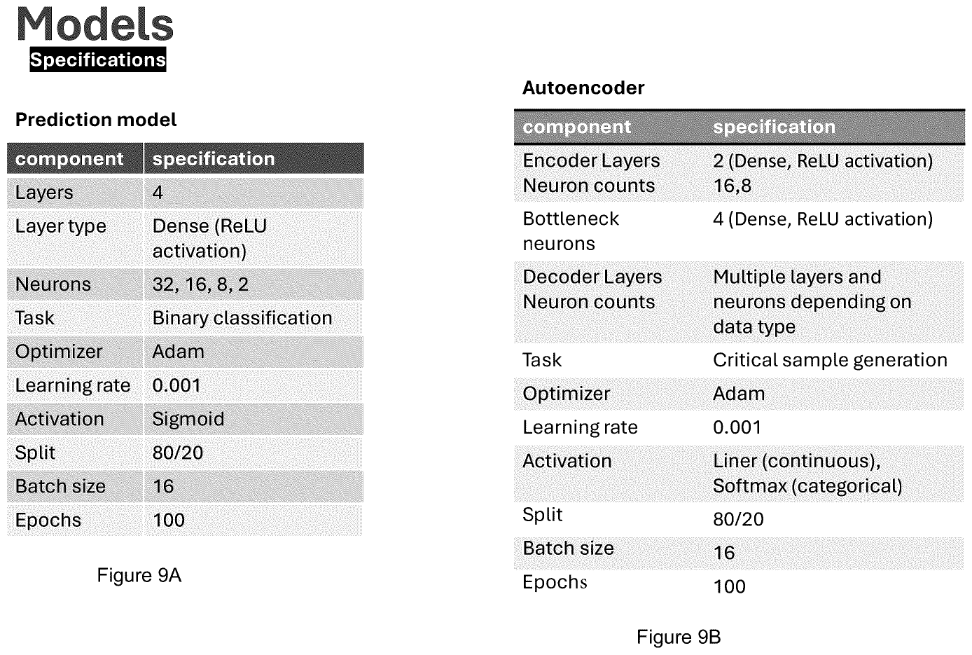

FIG. 9A shows components and specification of the classifier model, under an embodiment.

FIG. 9B shows components and specification of the autoencoder, under an embodiment.

The models described above may be used to provide counterfactual examples. Under an embodiment, counterfactual explanations provide insights into why a model made a particular prediction by generating hypothetical yet realistic instances that lead to a different prediction.

Example: A machine learning model predicts the probability of heart disease based on various health factors such as age, cholesterol levels, blood pressure, and exercise habits. A user inputs his information, and the model predicts a high likelihood of heart disease for him.

A counterfactual explanation generates a hypothetical scenario where the user's cholesterol levels are within a healthier range, their blood pressure is lower, and they engage in regular exercise. By making such lifestyle changes, the user's risk of heart disease could be significantly reduced.

A counterfactual explanation provides insights into hydration recommendations by showing alternative scenarios.

### Mathematical Framework:

Let X represent current physiological/environmental inputs, f(X) the model prediction, and X_cf the counterfactual input. Generate X_cf such that f(X_cf)=Euhydration and ∥X-X_cf∥ is minimized. Generating a counterfactual input X_{cf} such that the model's prediction f(X_{cf}) indicates a euhydrated state, while minimizing the difference between the original input X and X_{cf}, is indeed formulated as a minimization problem. The objective is to find the smallest possible change to the input features that results in the desired prediction outcome. This ensures that the system finds the smallest possible adjustment to the user's physiological, behavioral, or environmental features that would transition their hydration status from dehydration to euhydration.

### Mathematically:

$${min}_{{X}_{cf}} ‖ X - {X}_{cf} ‖ subject to f ( {X}_{cf} ) = Euhydration$$

Here, ∥X-X_{cf}∥ represents a distance metric (such as Euclidean distance) that quantifies the similarity between the original input X and the counterfactual input X_{cf}. The constraint f(X_{cf})=Euhydration ensures that the counterfactual input leads to a prediction of the euhydrated state.

To solve this optimization problem, one common approach involves iterative methods that adjust X_{cf} to minimize the distance ∥X-X_{cf}∥ while satisfying the prediction constraint. This process often utilizes gradient-based optimization techniques, where the gradients of the model's prediction with respect to the input features guide the adjustments needed to achieve the desired output. For instance, the DiCE (Diverse Counterfactual Explanations) framework approaches this by setting up the generation of counterfactual explanations as an optimization problem, aiming to find inputs that yield the desired prediction with minimal changes from the original input.
Example:
Current Input: User drank 500 mL, temperature 35° C., HR elevated.
Prediction: Risk of hyperhydration.
Counterfactual: If the user had reduced intake by 200 mL, hydration would remain optimal.

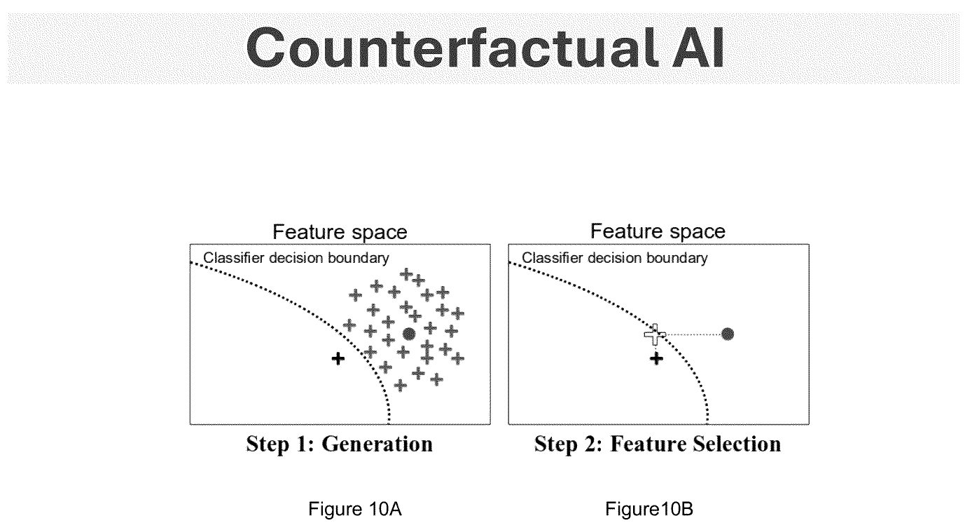

FIG. 10A shows that the counterfactual technique generates multiple intervention pathways, however, not all of them are feasible for the user to follow. For example, suggesting a patient with kidney disease to drink 500 ml water every hour might not be a feasible intervention.

FIG. 10B shows that of all the intervention plans generated in 10A, the method selects the most feasible one that requires minimal change/minimum number of feature change etc. and reflects the user's choice.

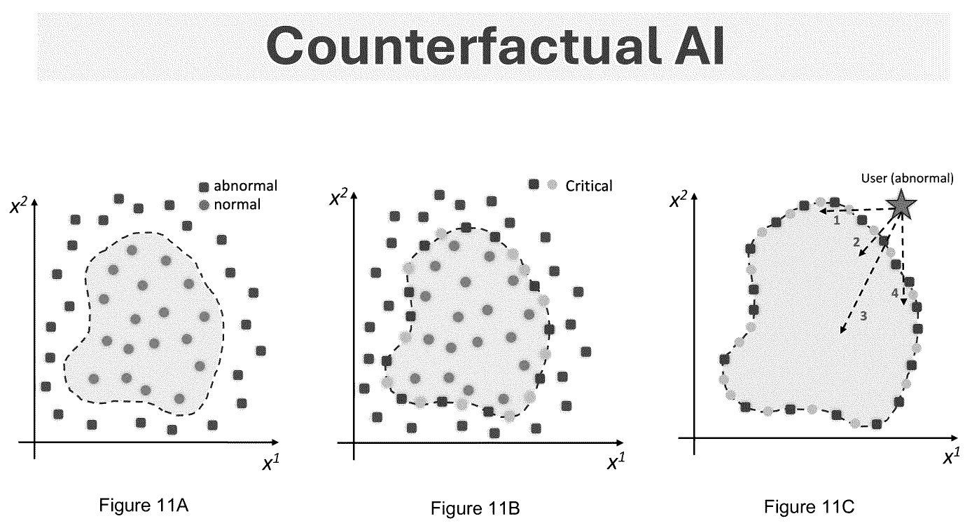

FIG. 11A shows dehydrated (red square) and hydrated (green dot) data with respect to a classifier space boundary generated by the neural net classifier described above.

FIG. 11B shows adversarial hydrated (yellow dot) and dehydrated (blue square) data that are generated using the autoencoder described above. These adversarial points represent and new classifier boundary space.

FIG. 11C illustrates use of the new classifier boundary space to identify an abnormal state of a user (purple star) and to provide the user with counterfactual information sufficient to transition the user from a dehydrated to a hydrated state.

Under an embodiment, data is collected using one or more of the following sensors:
Smart Wearable Watch: Monitors heart rate (HR), electrodermal activity (EDA), accelerometer data, and blood volume pulse (BVP).
Smart Water Bottle: Tracks fluid intake and identifies the type of fluid consumed (water, electrolyte drink, etc.).
Environmental Sensors: Logs external temperature and humidity.

Lab data: Collected in a controlled environment simulating heat exposure⇒this will work as a ground truth to train our model. Lab data will be collected in a controlled heat exposure environment to serve as ground truth for training the HydroGuard AI model. Physiological data from wearable sensors (heart rate, HRV, EDA, skin temperature, and BVP) will be recorded. Behavioral data, including fluid intake (volume and type) and activity levels, will be tracked using smart water bottles and motion sensors. Environmental factors such as ambient temperature, humidity, and radiant heat load will be monitored. Clinical hydration markers (body weight changes, urine specific gravity, and blood biomarkers) will be measured to validate hydration status. Data will be collected in three phases: baseline (euhydration), dehydration (heat exposure+mild exercise), and rehydration (fluid intake monitoring) to create a robust dataset for AI training.

Under an embodiment, target populations include Individuals with CKD, heart conditions, high-risk dehydration issues, fitness enthusiasts, and individuals exposed to extreme heat.

Under an embodiment, collected data includes Physiological signals, fluid intake patterns, activity levels, and environmental data.

Under an embodiment, a supervised machine learning model is trained to predict hydration states: Hypohydration, Euhydration, and Hyperhydration. This method implements Multi-Task Learning to predicts hydration levels and generate personalized recommendations simultaneously. The method comprises Counter Factual Based Supervised training and uses the following features:
Physiological data: Heart rate variability, skin conductance, and temperature;
Behavioral data: Activity levels, fluid intake patterns; and
Environmental data: Temperature, humidity.

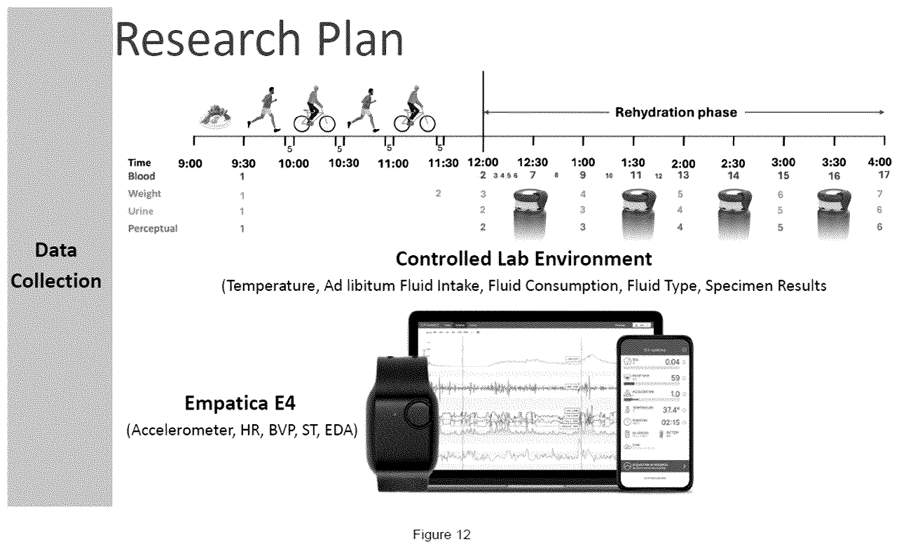

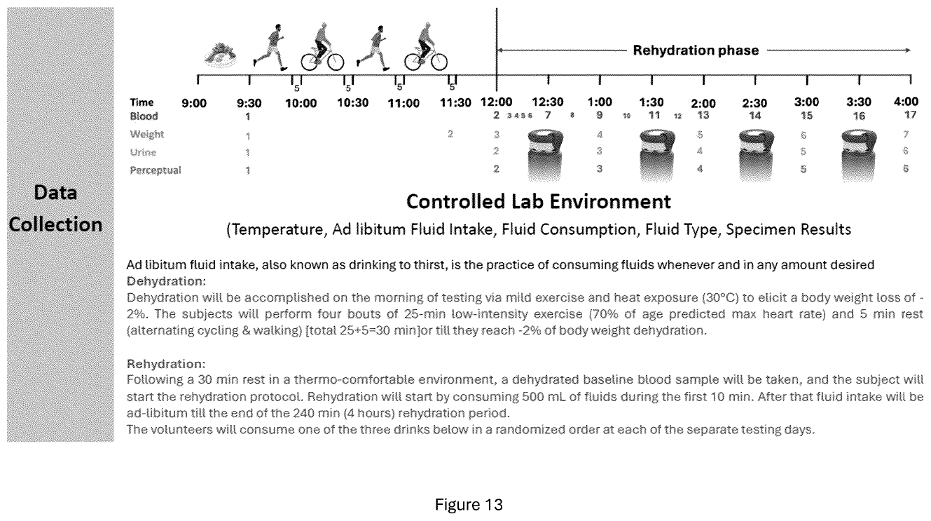

FIGS. 12 and 13 show data collection for purposes of training the model.

Here is the entire protocol of the in lab data collection process which was summarized in FIGS. 12 and 13

### Screening

During the screening visit, subjects will read the consent form discuss any concerns and address questions. After signing the consent from height and weight will be recorded, the medical health history and PAR-Q questionnaire form will be filled out. If the volunteer is eligible the experiments will be scheduled, and a frozen meal will be provided to be consumed as the dinner before the first experiment. More meals will be provided after the 1^{st} and 2^{nd} study for the next experiments.

### Protocol

Subjects will visit the lab after at least 10 hours of fasting (from food and beverages other than water), and a urine sample will be collected to ensure a proper hydration state. The urine sample will be analyzed for specific gravity (USG). If USG is below 1.020, the method proceeds with the protocol. If USG is >1.029, the testing will be canceled. If USG is between 1.020 and 1.029, the volunteer will consume 16 oz of water and another urine sample will be collected within 30 min. The protocol will be canceled unless USG is below 1.020 at the next sample of if the volunteer cannot provide an additional sample

After the hydration assessment, the subjects will be equipped with an Empatica E4 wristband (https://www.empatica.com/research/e4/) for physiological signal collection and will consume a light breakfast (i.e., a bagel with cream cheese and juice). A certified phlebotomist will insert an intravenous catheter to an antecubital vein and a blood sample will be taken after 20 min for baseline at seating position. Subjects will void their bladders (if needed) and a body weight will be taken.

### Dehydration:

Dehydration will be accomplished on the morning of testing via mild exercise and heat exposure (30° C.) to elicit a body weight loss of −2%. The subjects will perform four bouts of 25-min low-intensity exercise (70% of age predicted max heart rate) and 5 min rest (alternating cycling & walking) or till they reach-2% of body weight dehydration.

### Rehydration:

Following a 30 min rest in a thermo-comfortable environment, a dehydrated baseline blood sample will be taken, and the subject will start the rehydration protocol. Rehydration will start by consuming 500 mL of fluids during the first 10 min. After that fluid intake will be ad-libitum till the end of the 240 min rehydration period.

The volunteers will consume one of the three drinks below in a randomized order at each of the separate testing days.

### Drinks:

Water
Liquid I.V.® hydration multiplier
Liquid I.V.® hydration multiplier sugar free

All drinks will be enriched with 10 g of deuterium oxide isotope per liter of fluid (²H₂O, also known as D₂O) to be able to estimate fluid absorption.

### Measurements

All measurements will be performed at one of the test rooms at our lab facility that boarders the room where participants can relax and wait out the end of the study day. Duration of taking blood and urine samples will take 30-90 seconds each, and perceptual data will take 15-30 seconds per time point to be reported. To allow to perform all measurements study days will take up to 7 hours. Therefore, participants will be moved in a separate space after performing the dehydration protocol for self-study or watching video that includes a chair and table to work on and a couch to relax.

### Blood Samples (17 Per Trial, 90 Second Per Draw, a Total of 24 Minutes):

A total of seventeen blood samples (2 mL) will be collected on the euhydrated baseline (−150 min), dehydrated baseline (0 min), and at 5, 10, 15, 20, 30, 45, 60, 75, 90, 105, 120, 150, 180, 210, and 240 min of the rehydration period to be analyzed for blood 2H concentration. Also, seven blood samples (10 mL) at euhydrated baseline (−150 min) and during rehydration 0, 30, 60, 120, 180, and 240 min blood samples will be analyzed for glucose, osmolality, total plasma protein, hematocrit, hemoglobin (for plasma volume changes), sodium, potassium, chloride and copeptin (as a marker of arginine vasopressin, and therefore indicating water reabsorption by the kidney).

### Urine Samples and Bodyweight (6 Per Trial, 2 Minutes Per Sample, a Total of 12 Minutes);

Urine samples will be collected at euhydrated baseline (−150 min) and during rehydration at 0, 60, 120, 180, and 240 min. All samples will be analyzed for urine osmolality, specific gravity, volume, sodium, and potassium.

### Perceptual Data (6 Per Trial, 15-30 Seconds Per Combined Set of Scales, a Total of Maximal 3 Minutes):

Thirst, stomach fullness, and taste, assessed via a visual analog scale will be collected at euhydrated baseline (−150 min), dehydrated baseline (0 min), and at 60, 120, 180, and 240 min of the rehydration period.

### Physiological Signals (Continuous Data Collection Throughout the Trial, a Total of 7 Hours):

Participants will wear the wearable wristband before the beginning of the study, signals will be recorded during the study and the device will be removed once the trial is over for the day.

After completing all the trial each subject will perform a body composition assessment via dual x-ray absorptiometry. Female subjects will first perform a pregnancy test prior to the measurement. If test is positive no scan will be performed.

### Statistics:

Data will be presented as means and standard deviations unless non-normally distributed. To identify differences results, two-way (time-by-trial) repeated measures ANOVA will be employed or its non-parametric counterpart. Pair-wise differences will be evaluated using Tukey's post hoc procedure or a Wilcoxon signed rank test. For the purpose of hypothesis testing, the 95% level of confidence was predetermined as the minimum criterion to denote a statistical difference with P≤0.05.

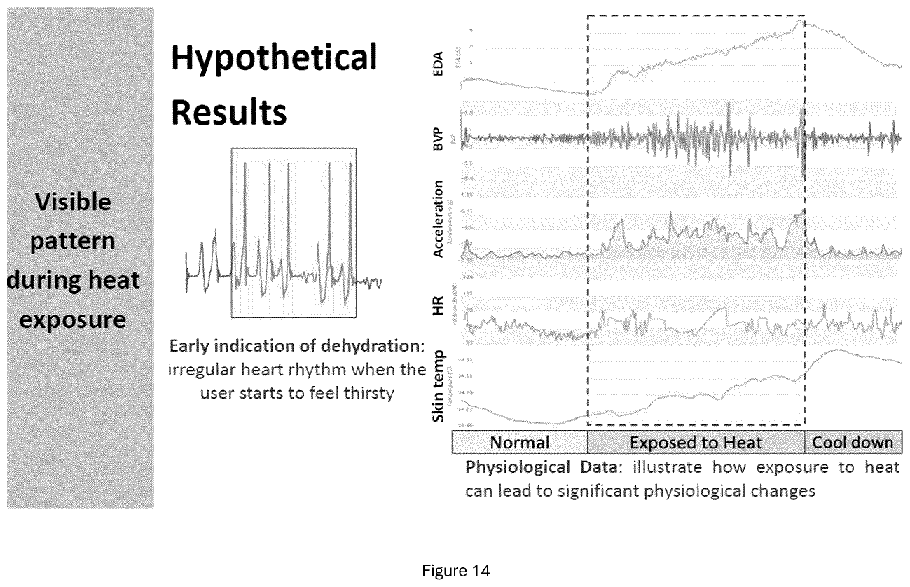

FIG. 14 shows how exposure to heat may cause significant physiological changes.

Various embodiments of the systems and methods described above are set forth below.

In one embodiment: Real-Time Hydration Monitoring

### Components:

Wearable Device: Monitors physiological parameters such as heart rate, skin conductance, and activity levels.
Smart Water Bottle: Tracks fluid intake volume and identifies the type of fluid consumed.

### Functionality:

Data Collection: The wearable device and smart bottle continuously gather real-time data on the user's physiological state and hydration habits.
Hydration Status Assessment: Analyzes collected data to determine the user's current hydration level.
User Alerts: Provides immediate notifications prompting the user to consume specific fluids to maintain optimal hydration.
   In another embodiment: Enhanced Personalization via Mobile Application

### Components:

Mobile Application: Serves as an interface for user input and personalized feedback.
Wearable Device and Smart Water Bottle.
Counterfactual Explanations: The app explains alternative actions to maintain hydration (Example: If a user plans to drink 500 mL of water before a run, the system may suggest drinking only 300 mL to avoid overhydration).

### Functionality:

User Input Integration: Allows users to input additional information such as planned physical activities, dietary preferences, and existing medical conditions.
Contextual Analysis: Combines user-provided data with sensor readings to refine hydration status assessments.
Tailored Recommendations: Delivers customized hydration advice considering both real-time sensor data and user-specific context.
   In another embodiment: Advanced Multi-Modal System for Long-Term Hydration Management

### Components:

Wearable Device: Enhanced with additional sensors, including Galvanic Skin Response (GSR) for stress monitoring, accelerometer, environmental sensors.
Continuous tracking of external conditions like temperature and humidity.

### Smart Water Bottle and Mobile Application

### Functionality:

Comprehensive Data Collection: Gathers extensive physiological and contextual data, including stress levels, to improve hydration status predictions. Provides more precise hydration predictions by incorporating diverse data sources.
Offers long-term hydration insights, allowing users to adjust strategies over time (e.g., tailoring hydration needs based on historical patterns and environmental trends).
Enables both real-time recommendations and long-term behavioral adjustments for sustained hydration management.
Counterfactual Reasoning: Utilizes AI algorithms to analyze past hydration behaviors and outcomes, providing insights on how different actions could have led to better hydration management.
Educational Feedback: Offers users actionable advice on preventing future hydration imbalances based on historical data analysis.
Practical implications of the systems and methods described herein include:
Personalized Hydration Guidance: Tailored recommendations for different demographics, including athletes, outdoor workers, and individuals with chronic conditions (e.g., CKD).
Prevention of Complications: Avoids dehydration-related risks such as heat stroke, kidney malfunction, and electrolyte imbalance.
Continuous Monitoring: Provides insights throughout the day, ensuring consistent hydration management.
Immediate Alerts: Users receive real-time guidance to prevent hydration-related complications.
Healthcare Integration: Can integrate with health systems for remote patient monitoring.
Under an embodiment, the systems and methods described herein collects variable physiological responses across users and environments, seamlessly combines multi-source data into one cohesive system, ensures the system operates continuously without draining device batteries, and educates users on the importance of hydration monitoring to drive adoption.

Benefits of the systems and methods described herein include the following:
Health Benefits:
Reduces the risk of dehydration-related illnesses like edema or electrolyte imbalance.
Improves quality of life for individuals in heat-prone regions or high-risk populations.

### Scalability:

Applicable to diverse user groups, from athletes to individuals with chronic conditions.
Even healthy individuals can also use it to track their hydration status to stay hydrated during the summer.
The system not only capable of detection hydration it can also alert people of being overhydrated which can be dangerous for kidney disease patients.

### Innovation in Hydration Management:

First-of-its-kind system integrating AI, wearable tech, and counterfactual reasoning for proactive hydration management.

The following disclosure presents a new method for identifying most diverse counterfactual treatments out of a given set of feasible counterfactuals.

### Graph-Based, Preference-Aware Diverse Counterfactual Explanations for Hydration Recourse

This method presents a framework for generating diverse, personalized counterfactual (CF) recommendations that guide a user from a dehydrated state to an adequately hydrated state. Given a predictive model trained on hydration-related features (e.g., fluid intake, activity level, sweat loss, ambient temperature and humidity), standard counterfactual methods can return multiple feasible interventions that would flip the model's prediction. However, these counterfactuals are often redundant and may ignore user preferences (e.g., willingness to drink more but not reduce training). Given a set of ‘n’ feasible counterfactual treatments, a method is proposed for identifying ‘k’ most diverse counterfactual treatments.

A graph-based selection method is proposed that operates on a set of pre-generated feasible counterfactuals. First, this approach models the problem of representing counterfactual data points and their relationship as a complete graph where each counterfactual is represented as a node in a complete graph, with edge weights encoding behavioral diversity (distance in intervention space), and node utilities encoding user preferences over which features they are more willing to modify. The approach then selects a subset of kcounterfactuals by maximizing a joint objective that balances diversity and preference alignment, solved via a simple greedy max-dispersion algorithm. The method produces a small set of distinct, realistic, and user-aligned rehydration strategies.

### 1. Introduction

Hydration dynamics are influenced by several factors such as:
Fluid intake (how much and when a person drinks),
Physical activity and sweat rate,
Ambient temperature and humidity,
Individual physiology (total body water, body mass).

A predictive model trained on such data can classify an hourly state as adequately hydrated or dehydrated, and even estimate risk ahead of time.

However, when the model predicts dehydration, simply saying “you are dehydrated” is not enough. The method answers the following question:

“What can this specific person do now (or over the next hours) to become adequately hydrated again?”

Counterfactual explanations give candidate interventions: “Increase water intake by 300 ml this hour”, or “Reduce exercise intensity and drink slightly more”. But in practice:
There may be many feasible counterfactuals.
Users differ in preferences and constraints:
   An athlete may refuse to lower training intensity but can drink more.
   An office worker may prefer moving more rather than drinking a lot (because of bathroom breaks).
Showing many near-duplicate suggestions (e.g., “drink 500 ml vs 520 ml”) is not useful because it can lead to reduced engagement of the users in utilizing the intelligent system (an app that prompt the person how to remain hydrated in real-time).

The approach therefore needs a way to:
1. Start from a set of valid counterfactuals (all of which flip the prediction from for example dehydrated to adequately hydrated),
2. Model how different they are as interventions,
3. Encode user preferences over which features are more acceptable to change,
4. Select k counterfactuals that are:
   All valid (flip the model's initial outcome),
   Mutually diverse,
   And aligned with user preferences.

This is exactly what our graph-based, preference-aware selection method does.

### Context:

The method implements a known predictive AI model f(for hydration status, but generally any health/behavior outcome). Under an embodiment, the AI predictive model f comprises the model set forth in FIG. 9A above but differing predictive models may be used.
For a “bad” prediction (e.g., dehydrated), the approach can generate many feasible counterfactuals-different combinations of behavior changes (e.g., drink more, reduce activity, change water intake timing) that would flip the prediction to rehydrated. One approach to generate these feasible counterfactuals is to generate data points that are close to the decision boundary between the two classes (e.g., between dehydrated and hydrated) using an auto-encoder method
Not all of these are helpful. Many can be redundant or conflict with the user's preferences (e.g., user does not want to reduce workout intensity but is willing to drink more fluid). The invention:
1. Treats each feasible counterfactual as a node in a graph.
2. Connects every pair of nodes with an edge whose weight measures how different (diverse) those two counterfactuals are.
3. Assigns each node a preference-based utility score, derived from the user's stated preferences over which features they are more willing to change.
4. Selects exactly k counterfactuals (e.g., 3-5 recommendations) that jointly:
   are maximally diverse from each other; and
   are aligned with user preferences.

This is formulated as a graph-based optimization problem over the set of feasible counterfactuals.

Steps 1, 2, and 3 are graph modeling (constructing a graph and specifying its parameters) while step 4 represents an optimization over the constructed graph.

### Notation

X_{T}∈ [symbol] ^{d}: factual instance (e.g., current hydration state features)·
f: [symbol] ^{d}→ [symbol] ^{c}: trained classifier/AI model
ŷ_{T}=arg max f(X_{T}): predicted class using X_{T} (e.g., “dehydrated”)·
[symbol] ={C₁, . . . , Cₙ}: set of feasible counterfactuals, each:
   1. flips the class to a desired outcome (e.g., “rehydrated”)
   2. is within a minimal distance threshold from X_{T}
   3. is realistic (constrained to the data distribution)
d_{div}(Cᵢ, Cⱼ): diversity distance between two counterfactuals
P=[p₁, . . . , p_{d}]: user preference vector over features
u(Cᵢ): preference-based utility of counterfactual Cᵢ
G=(V, E): graph where nodes/vertex is V= [symbol] , edges weighted by diversity
k: desired number of counterfactuals to present
S⊆ [symbol] , |S|=k: selected subset of counterfactuals
   2. Problem Formulation
   2.1 Hydration Dataset and Model

Let D={(X₁, y₁), (X₂, y₂), . . . , (Xₙ>yₙ)} be a dataset of hourly hydration states (‘hourly’ here is used as an example; in reality, these data points can represent any time resolution such as minute-level, hourly, daily, weekly and even monthly), where each:
Xᵢ∈ [symbol] ^{d} is a feature vector containing, for example:
   intake_ml—fluid intake in the hour (mL)—The total volume of fluids consumed by the individual during a given one-hour interval, expressed in milliliters (mL). This may include water and other beverages that contribute to body water.
   activity_met—activity level (METs)—MET itself is a unit. Formal definition: One MET is defined as the consumption of 3.5 ml of oxygen per kilogram of body weight per minute (3.5 ml/kg/min) or 1 kcal/kg/hour. It measures activity intensity relative to resting, which is 1MET
   sweat_loss_lph—sweat loss (L/hour),
   ambient_temp_c—ambient temperature (° C.),
   humidity_rh—relative humidity (%),
   tbw_1—total body water (L),
   time-of-day features (hour_of_day, etc.).

For example, a wrist-worn device (e.g., smartwatch or fitness band) equipped with a 3-axis accelerometer and, optionally, a heart-rate sensor can be used to estimate energy expenditure and derive the activity level in METs on an hourly basis, while integrated temperature and humidity sensors (i.e. integrated in the wrist-worn device or other device worn by a user) or a nearby environmental node provide ambient temperature and relative humidity. Fluid intake in milliliters per hour may be captured by a “smart bottle” or cup with an inline flow sensor or load cell that records volume and timestamps each drinking event, or alternatively via user logging in a smartphone application. Sweat loss can be estimated using a combination of activity, temperature and humidity, optionally calibrated using sweat patches or body-mass measurements in controlled settings. Total body water may be initialized from anthropometric data or intermittent bioelectrical impedance measurements (e.g., from a smart scale), and then updated by a software water-balance model using the measured intake and loss terms. All sensors or devices communicate time-stamped data to a processing unit (e.g., phone or cloud service), which aggregates values into hourly windows and computes the derived hydration features. Under an embodiment all such data may be obtained using smart sensors/devices worn or used by user in real world or laboratory conditions. As indicated above, the predictive model may run on a remote server or on a user's mobile device.
yᵢ∈{0,1} is the hydration label:
   0=dehydrated/inadequate,
   1=adequate.

Let, f: [symbol] ^{d}→[0,1] be a probabilistic classifier where:

$$f ( X ) = P ( y = 1 \vert X ) ,$$

and the approach predicts:

$$\hat{y} ( X ) = \{ \begin{matrix}1 & if f ( X ) \geq τ , \\ 0 & otherwise ,\end{matrix}$$

for threshold τ (e.g. 0.5 or tuned).

The approach focuses on a factual instance X_{T} such that, ŷ(X_{T})=0 (i.e., the model predicts the user is dehydrated).

### 2.2 Counterfactual Explanations for Hydration

A counterfactual explanation C for X_{T} is a modified feature vector
C∈ [symbol] ^{d} satisfying:
1. Interventional: f(C)≥τ⇒ŷ(C)=1 (the prediction flips to “adequate hydration”). [manually established]
2. Minimal: d(C, X_{T})≤∈ (the changes from current behavior are small).
This is a design parameter chosen by the system designer (e.g., based on domain knowledge or validation experiments)
3. Realistic: C lies within a realistic physiological/behavioral region (e.g., intake within human limits, METs plausible, etc.). example, for the intake_ml feature, the system may restrict hourly fluid intake to a range of about 0-1500 mL/hour, with typical comfortable values for most adults between and higher values flagged as potentially unrealistic or clinically risky. For the activity_met feature, the system may constrain MET values to approximately 1.0-12.0, where 1.0 corresponds to resting metabolism, 1-3 METs correspond to light activities (sitting, slow walking), 3-6 METs to moderate activities (brisk walking, easy running), and values up to ~10-12 METs are reserved for vigorous exercise in trained or athletic individuals. Counterfactuals that fall outside these bounds are rejected or projected back into the allowable range.
4. Actionable: Only actionable features (e.g., intake, activity, timing) are allowed to change; non-actionable ones (e.g., height, body mass) are kept fixed.

The approach assumes some counterfactual generator (gradient-based, NiCE-like, ExACT, heuristic search, etc.) has already produced a feasible set:

$$𝒞 = \{ {C}_{1} , {C}_{2} , … , {C}_{n} \} ,$$

where each Cᵢ satisfies the four properties above.

Our contribution is not a new CF generator, but a method for selecting k counterfactuals from [symbol] that are diverse and preference-aware.

It is noted that the EXACT approach is described in PCT Application No. PCT/US24/45606, filed Sep. 6, 2024, which is incorporated herein by reference in its entirety.

### 2.3 User Preference Vector

Different users tolerate different changes:
An athlete might say: “Don't reduce my training, I'll drink more.”
An office worker might say: “I can walk more, but I don't want to chug water all day.”

The approach models this with a preference vector:

$$P = [ {p}_{1} , {p}_{2} , … , {p}_{d} ] ,$$

where:
pₗ≥0 encodes willingness to modify feature l,
larger pₗ=more acceptable to change this feature,
for non-actionable features, the approach can set pₗ=0 and disallow changes.
The feature has a scale of 0-1. Elements of P can be obtained in practice from a short questionnaire or user interface (e.g., sliders indicating “willing to drink more” vs. “willing to reduce exercise”), or specified by a clinician or system designer; the algorithm itself only assumes that these non-negative weights are provided.
For example:
intake_ml: pᵢₙₜₐₖₑ=1.0 (user is happy to change intake),
activity_met: p_{activity}=0.2 (user dislikes modifying activity),
non-actionable features: pₗ=0.

### 3. Graph-Based Model Over Counterfactuals

The approach now defines a graph G with a set of vertices V over the set of feasible counterfactuals [symbol] to capture diversity.

### 3.1 Nodes and Edges

The approach defines a complete graph:

$$G = ( V , E )$$

where:
V= [symbol] ={C₁, . . . , Cₙ}: each node is a feasible counterfactual.
For each pair i≠j, there is an edge eᵢⱼ between Cᵢ and Cⱼ.

### 3.2 Edge Weights: Diversity Distances

The approach defines an edge weight wᵢⱼ as a diversity distance between two counterfactuals (Cᵢ, Cⱼ):

$${w}_{ij} = {d}_{div} ( {C}_{i} , {C}_{j} ) .$$

A simple and effective choice is a weighted L1 distance on selected actionable features:

$${d}_{div} ( {C}_{i} , {C}_{j} ) = \text{?} {α}_{l} \vert {C}_{i}^{l} - {C}_{j}^{l} \vert ,$$

The weighted L1 distance is a metric used to calculate the distance between two vectors by summing the absolute differences of their components, with each component difference multiplied by a specific weight. Under an embodiment, an L2 norm can also be used.

where:
A is the set of actionable/intervention features (e.g., intake_ml, activity_met, timing),
   αₗ>0 are feature-specific weights to emphasize certain dimensions as more important sources of diversity (e.g., treat large changes in METs as more “different” than small changes in intake). The scale of αₗ is 0-1. The values are manually determined.

### Interpretation:

Large wᵢⱼ⇒Cᵢ and Cⱼ represent very different intervention strategies.
Small wᵢⱼ⇒interventions are similar (redundant to show both).

Note: In our working example, the approach uses raw distances as the diversity measure (no kernel or exponential transformation). However, the framework is more general: in other settings the diversity term could be defined via a similarity kernel (e.g., Gaussian/RBF) or other nonlinear transformations of the pairwise distances. However, under an embodiment, the following may be used:

$${w}_{ij} = exp ( - \frac{{‖ {C}_{i} - {C}_{j} ‖}^{2}}{2 {σ}^{2}} )$$

where σ controls the locality scale. In that case, diversity is dᵢⱼ=1−wᵢⱼ so pairs that are far apart in feature space contribute larger diversity, while near-duplicate counterfactuals contribute near-zero diversity.

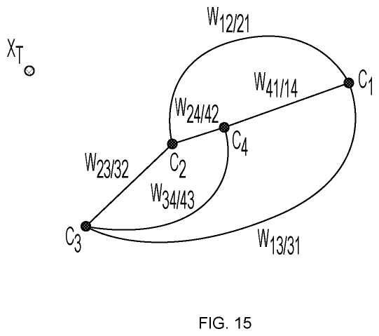

FIG. 15 illustrates a graph representation of candidate counterfactuals for a factual instance X_{T}. Each node C₁ . . . . C₄ denotes a feasible counterfactual, and each edge is labelled with the pairwise diversity weights wᵢⱼ/wⱼᵢ capturing how different the corresponding interventions are when moving from Cᵢ to Cⱼ. This complete graph is the basis for selecting a small, diverse subset of counterfactuals to recommend.

### 3.3 Node Utilities: Preference-Aware Cost

To encode how comfortable a user is with a given counterfactual, the approach defines a preference-based utility:

$$u ( {C}_{i} ) = - {\sum }_{l = 1}^{d} {p}_{l} \vert {C}_{i}^{l} - {X}_{T}^{l} \vert$$

This is a weighted L1 distance, under an embodiment. The weighted L1 distance is a metric used to calculate the distance between two vectors by summing the absolute differences of their components, with each component difference multiplied by a specific weight. Under an embodiment, an L2 norm can also be used.

$$\vert {C}_{i}^{l} - {X}_{T}^{l} \vert :$$

magnitude of change in feature l,
pₗ: user's willingness to change feature l,
negative sign: a smaller total preference-weighted change⇒higher utility.
So
High utility→changes are small and mostly in preferred features (e.g. more water intake).
Low utility→requires big changes in features user dislikes modifying (e.g. cutting training volume).

### 4. Optimization Objective

The approach wants to select a subset:

$$S ⊆ 𝒞 , \vert S \vert = k$$

of exactly kcounterfactuals that:
1. Are mutually diverse (high average pairwise distance),
2. Are individually acceptable (high utility).

The approach formalizes this with a joint objective:

$${max}_{S ⊆ 𝒞 , \vert S \vert = k} J ( S ) = λ {\underbrace{{{\sum }_{{C}_{i} , {C}_{j} ∈ S}}_{i < j} {w}_{ij}}}_{diversity} + ( 1 - λ ) {\underbrace{{\sum }_{{C}_{i} ∈ S} u ( {C}_{i} )}}_{preference}$$

where:
λ∈[0,1] controls trade-off:
λ→1: focus on diversity,
λ→0: focus on user preferences.
This is a max-dispersion-plus-utility problem over the counterfactual graph.

### 5. Proposed Algorithm

The system operates in three phases:
1. Phase 1—Feasible counterfactual generation
   (any CF generator; not part of the novelty here).
2. Phase 2—Graph & utilities
   Compute diversity distances wᵢⱼ and utilities u(Cᵢ).
3. Phase 3—Greedy selection
   Select k CFs using a greedy algorithm that approximates the objective.
   5.1 Phase 1—Generate Feasible Counterfactuals

The approach treats this as a black box. Any existing method (DiCE, gradient-based optimization, search over simulator, etc.) that returns a set, [symbol] ={C₁, . . . , Cₙ} such that each Cᵢ is:
class-flipping: ŷ(Cᵢ)=1,
close to X_{T},
realistic.

```
Algorithm 1: Generation of Realistic Counterfactual Candidates
  Input: Factual dehydrated instance X_{T};
  Trained classifier ƒ;
  Desired target class y_{target} (adequate = 1);
  Maximum allowed distance ε from X_{T};
  Realism model or constraint [symbol]  ᵣₑₐₗᵢₛₘ;
  Maximum number of candidates Nₘₐₓ.
  Output: Set of counterfactuals Cₛₑₜ = {C₁,...,Cₙ}
  Cₛₑₜ ← Ø;
  while |Cₛₑₜ| < Nₘₐₓ do
  | C_{cand} ← PROPOSECANDIDATE(X_{T},ƒ,y_{target});
  | if ƒ (C_{cand}) ≥ τ; / / Classifier predicts target class
  |  then
  | | if DISTANCE(C_{cand}, X_{T}) ≤ ε then
  | | | if ISREALISTIC(C_{cand}, [symbol]  ᵣₑₐₗᵢₛₘ) then
  | |_ |_ |_ Cₛₑₜ ← Cₛₑₜ ∪ {C_{cand}};
  |_
  return Cₛₑₜ;
```

### 5.2 Phase 2—Build Diversity Matrix & Utilities

The approach computes pairwise diversity weights and per-node utility.

```
Algorithm 2: BuildGraphAndUtilities
  Input: Feasible counterfactual set Cₛₑₜ = {C₁,...,Cₙ};
  Factual instance X_{T};
  User preference weights P = [p₁,...,p_{d}];
  Diversity distance function d_{div}(.,.).
  Output: Diversity matrix W[1..n,1..n];
  Utility vector U[1..n].
  for i ← 1 to n do
  | U[i] ← 0;
  | for l ← 1 to d do
  | | Δ← |Cᵢ[I] − X_{T}[I]|;
  | |_ U[i] ← U[i] − P[l] · Δ;
  |_
  for i ← 1 to n do
  | for j ← i + 1 to n do
  | | W[i,j] ← d_{div} (Cᵢ,Cⱼ);
  | |_ W[j,i] ←W[i, j];
  |_
  return W, U;
```

### 5.3 Phase 3—Greedy Diverse & Preference-Aware Selection

The exact optimization is combinatorial and NP-hard. We use a greedy max-dispersion algorithm, which is simple and effective for small k (e.g., 3-5 recommendations). Our greedy selection is a known 2-approximation for the max-sum dispersion objective when the diversity distance is a metric and the approach only imposes a cardinality constraint.

```
Algorithm 3: SelectDiversePreferenceAwareCFs
  Input: Feasible counterfactual set Cₛₑₜ = {C₁,...,Cₙ}
  Diversity matrix W[1..n,1..n];
  Utility vector U[1..n];
  Number of counterfactuals to select k;
      Trade-off parameter λ ∈ [0,1].
      Output: Indices of selected counterfactuals S_{indices}.
  S_{indices} ← Ø;
  /* Step 1: Initialize with best − utility CF */;
  best_idx ← arg maxᵢ U[i];
  S_{indices} ← S_{indices} ∪ {best_idx}
  /* Step 2: Iteratively add CFs balancing diversity and utility */;
  while |S_{indices}| < k do
  | best_candidate ← None;
  | best_score ← −∞;
  | for i ← 1 to n do
  | | if i [symbol]   S_{indices} then
  | | | diversity_sum ← 0;
  | | | foreach j ∈ S_{indices} do
  | | | |_ diversity_sum ← diversity_sum + W [i,j];
  | | | score ←λ· diversity_sum + (1 −λ) · U[i];
  | | | if score > best_score then
  | | | | best_score ← score;
  | | | |_ best_candidate ← i;
  | | |_
  | |_
  |_ S_{indices} ← S_{indices} ∪ {best_candidate};
      return S_{indices};
```

### Intuition Behind Algorithm 3:

First, the approach picks the counterfactual that is most aligned with user preferences (max U).
Then, each new CF the approach adds must:
   be different from the ones already chosen (large diversity_sum),
   and still be reasonably aligned with preferences (utility term).
The trade-off is controlled by A.

### 6. Case Analysis: How the Algorithms Works

To make the method concrete, the approach analyzes a toy hydration scenario. Suppose:
Factual dehydrated instance X_{T} (intake_ml=100, activity_met=7.0) (high-intensity workout with low drinking)
The counterfactual generator returns four feasible CFs {C₁, . . . , C₄}:

| CF | intakeₘₗ | activity_{MET} | Interpretation |
|---|---|---|---|
| C₁ | 400 | 7.0 | Large extra drinking, same |
| | | | workout intensity |
| C₂ | 250 | 5.5 | Moderate drinking, moderate |
| | | | reduction in activity |
| C₃ | 150 | 4.0 | Small extra drinking, big |
| | | | reduction in activity |
| C₄ | 300 | 6.0 | Extra drinking, small |
| | | | reduction in activity |

All four CFs:
Flip the classifier to adequate hydration,
Are within an acceptable distance of X_{T},
Are physiologically realistic.

The approach wants to select k=2 CFs to present.

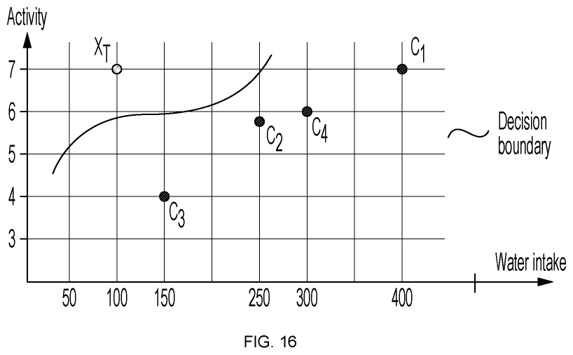

FIG. 16 illustrates a graph of counterfactual and XT feature vectors, under an embodiment.

### 6.1 User Preferences

User says: “I prefer drinking more water and I really dislike cutting my workout intensity.”

The approach encodes:
pᵢₙₜₐₖₑ=1.0 (very okay to change intake),
p_{activity}=0.2 (small weight⇒changes in activity are relatively more “painful”).
   Then:

$$u ( {C}_{i} ) = - ( {p}_{intake} \vert {Δ}_{intake} \vert + {p}_{activity} \vert {Δ}_{MET} \vert )$$

Compute utilities (difference from X_{T}=[100,7.0]):

| CF | intakeₘₗ | activity_{MET} | \|Δᵢₙₜₐₖₑ\| | \|Δ_{MET}\| | u(Cᵢ) |
|---|---|---|---|---|---|
| C₁ | 400 | 7.0 | 300 | 0 | −300 |
| C₂ | 250 | 5.5 | 150 | 1.5 | −150.3 |
| C₃ | 150 | 4.0 | 50 | 3 | −50.6 |
| C₄ | 300 | 6.0 | 200 | 1 | −200.2 |

So, u(C₃)>u(C₂)>u(C₄)>u(C₁), from a preference standpoint, C₃ is most comfortable (small intake change, but big hit to activity).

### 6.2 Diversity Distances

The approach defines a diversity distance:

$${d}_{div} ( {C}_{i} , {C}_{j} ) = \vert {Δ}_{intake} \vert + 50 \cdot \vert {Δ}_{MET} \vert$$

to emphasize that different activity patterns count heavily as “different interventions”.

| CF | intakeₘₗ | activity_{MET} | \|Δᵢₙₜₐₖₑ\| | \|Δ_{MET}\| | u(Cᵢ) |
|---|---|---|---|---|---|
| C₁ | 400 | 7.0 | 300 | 0 | −300 |
| C₂ | 250 | 5.5 | 150 | 1.5 | −150.3 |
| C₃ | 150 | 4.0 | 50 | 3 | −50.6 |
| C₄ | 300 | 6.0 | 200 | 1 | −200.2 |

### Compute Pairwise Distances:

$$d ( {C}_{1} , {C}_{2} ) = \vert 400 - 250 \vert + 50 \vert 7. - 5.5 \vert = 225$$

$$d ( {C}_{1} , {C}_{3} ) = \vert 400 - 150 \vert + 50 \vert 7. - 4. \vert = 400$$

$$d ( {C}_{1} , {C}_{4} ) = \vert 400 - 300 \vert + 50 \vert 7. - 6. \vert = 150$$

$$d ( {C}_{2} , {C}_{3} ) = \vert 250 - 150 \vert + 50 \vert 5.5 - 4. \vert = 175$$

$$d ( {C}_{2} , {C}_{4} ) = \vert 250 - 300 \vert + 50 \vert 5.5 - 6. \vert = 75$$

$$d ( {C}_{3} , {C}_{4} ) = \vert 150 - 300 \vert + 50 \vert 4. - 6. \vert = 250$$

As indicated above, FIG. 15 illustrates a graph representation of candidate counterfactuals for a factual instance X_{T}. Each node C₁ . . . . C₄ denotes a feasible counterfactual, and each edge is labelled with the pairwise diversity weights wᵢⱼ/wⱼᵢ capturing how different the corresponding interventions are when moving from Cᵢ to Cᵢ. This complete graph is the basis for selecting a small, diverse subset of counterfactuals to recommend.

6.3 Greedy Selection (λ=0.5, k=2) Using Algorithm 3

We set λ=0.5, giving equal weight to diversity and utility.

### Step 1—Initialization

Pick CF with best utility: ⇒max utility isu(C₃)=−50.6
So, the approach starts with: S={C₃}

### Step 2—Add Second Counterfactual

For each remaining candidate Cᵢ, compute:

$$score ( {C}_{j} ) = λ \cdot diversity\_sum + ( 1 - λ ) \cdot u ( {C}_{i} )$$

Here, diversity_sum=d_{div}(Cᵢ, C₃) and λ=0.5.

### Compute:

For C₁: diversity_sum=d(C₁, C₃)=400 & score=0.5·400+0.5. (−300.0)=50.0
For C₂: diversity sum=d(C₂, C₃)=175 & score=0.5·175+0.5. (−150.3)≈12.35
For C₄: diversity_sum=d(C₄, C₃)=250 & score=0.5·250+0.5. (−200.2)=24.9 score (C₁)=50.0>score (C₄)=24.9>score(C₂)≈12.35

So C₁ is selected.

Thus, the final selected subset: S={C₃, C₁}.

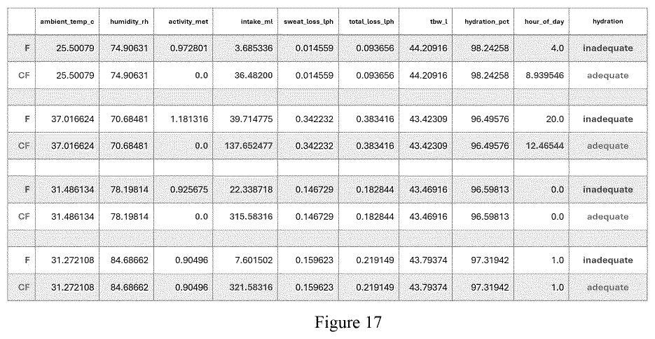

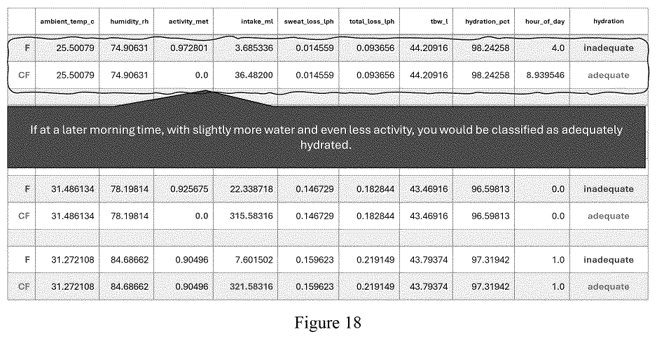

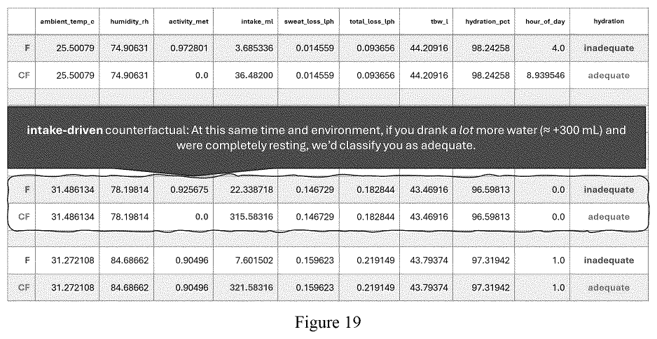

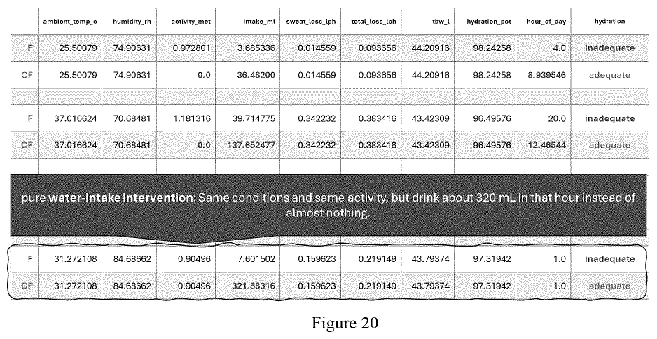

FIGS. 17-20 illustrates factual-counterfactual pairs, under an embodiment.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The systems, methods, and apparatuses for continuous monitoring of and effective management of hydration can be a component of a single system, multiple systems, and/or geographically separate systems. The systems, methods, and apparatuses for continuous monitoring of and effective management of hydration can also be a subcomponent or subsystem of a single system, multiple systems, and/or geographically separate systems. The components of systems, methods, and apparatuses for continuous monitoring of and effective management of hydration can be coupled to one or more other components (not shown) of a host system or a system coupled to the host system.

One or more components of the systems, methods, and apparatuses for continuous monitoring of and effective management of hydration and/or a corresponding interface, system or application to which the systems, methods, and apparatuses for continuous monitoring of and effective management of hydration is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the systems, methods, and apparatuses for continuous monitoring of and effective management of hydration can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems, methods, and apparatuses for continuous monitoring of and effective management of hydration and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems, methods, and apparatuses for continuous monitoring of and effective management of hydration and corresponding systems and methods include: microcontrollers with memory (such as electronically erasable programmable read only memory (EEPROM)), embedded microprocessors, firmware, software, etc. Furthermore, aspects of the systems, methods, and apparatuses for continuous monitoring of and effective management of hydration and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems, methods, and apparatuses for continuous monitoring of and effective management of hydration is not intended to be exhaustive or to limit the systems and methods to the precise forms disclosed. While specific embodiments of, and examples for, the systems, methods, and apparatuses for continuous monitoring of and effective management of hydration and corresponding systems and methods are described herein for illustrative purposes, various equivalent modifications are possible within the scope of the systems and methods, as those skilled in the relevant art will recognize. The teachings of the systems, methods, and apparatuses for continuous monitoring of and effective management of hydration and corresponding systems and methods provided herein can be applied to other systems and methods, not only for the systems and methods described above.

The elements and acts of the various embodiments described above can be combined to provide further embodiments. These and other changes can be made to the systems, methods, and apparatuses for continuous monitoring of and effective management of hydration and corresponding systems and methods in light of the above detailed description.

A method is described herein under an embodiment comprising one or more applications running on at least one processor for providing, collecting feature space data of a plurality of subjects using a plurality of sensors, wherein the feature space data comprises hydration state, wherein the hydration state comprises a hydrated state or a dehydrated state, generating a classifier space boundary, wherein the generating comprises training a neural network using information of the feature space data, wherein the trained neural network identifies the hydration state of a subject based on corresponding feature space data, training an autoencoder using information of the feature space data of the plurality of subjects and the trained neural network, using the autoencoder to generate an adversarial space boundary, wherein the generating comprises producing feature data points along a boundary between the hydrated state feature space data and the dehydrated state feature space data, receiving the feature space data of a first subject using the plurality of sensors, detecting a hydration state of the first subject using information of the first subject feature space data relative to the adversarial space boundary, computing the shortest distance between a feature vector of the first subject feature space data and a nearest interior of the adversarial space boundary, and recommending minimum changes to the first subject feature space data necessary to effect transition from a dehydrated state to a hydrated state.

In embodiments, the recommended minimum changes comprises modification of user behavior corresponding to targeted changes of the first subject feature space data.

In embodiments, the recommending comprises applying a gradient based optimization method to compute the shortest Euclidean distance between the feature vector of the first subject feature data to the nearest interior of the adversarial space boundary.

In embodiments, the optimization identifies smallest perturbations to variables of the first subject feature space data required to effect the transition.

In embodiments, the optimization prioritizes actionable features with the highest sensitivity to hydration state changes.

In embodiments, the actionable features comprise fluid intake and activity level.

In embodiments, the plurality of sensors includes wearable devices, smart objects, and mobile devices.

In embodiments, the feature space data comprises physiological signals.

In embodiments, the physiological signals include heart rate variability.

In embodiments, the physiological signals include electrodermal activity.

In embodiments, the physiological signals include skin temperature.

In embodiments, the feature space data comprises behavioral data.

In embodiments, the behavioral data includes fluid intake patterns.

In embodiments, the behavioral data includes activity levels.

In embodiments, the feature space data comprises environmental information.

In embodiments, the environmental information includes ambient temperature.

In embodiments, the environmental information includes humidity.

A system is described herein under an embodiment comprising one or more applications running on at least one processor of a remote server, the one or more applications communicatively coupled with a mobile device application running on a processor of a mobile device, the one or more applications configured to collect feature space data of a plurality of subjects using a plurality of sensors, wherein the feature space data comprises hydration state, wherein the hydration state comprises a hydrated state or a dehydrated state, generate a classifier space boundary, wherein the generating comprises training a neural network using information of the feature space data, wherein the trained neural network identifies the hydration state of a subject based on corresponding feature space data, train an autoencoder using information of the feature space data of the plurality of subjects and the trained neural network, use the autoencoder to generate an adversarial space boundary, wherein the generating comprises producing feature data points along a boundary between the hydrated state feature space data and the dehydrated state feature space data. The mobile device application receives information of the adversarial space boundary from the one or more applications, the mobile device application configured to receive the feature space data of a first subject using the plurality of sensors, detect a hydration state of the first subject using information of the first subject feature space data relative to the adversarial space boundary, compute the shortest distance between a feature vector of the first subject feature space data and a nearest interior of the adversarial space boundary, recommend minimum changes to the first subject feature space data necessary to effect transition from a dehydrated state to a hydrated state.

1. A method comprising,
receiving a feature vector for a plurality of subjects, wherein a feature vector comprises feature data elements of a subject;
applying a predictive model to the feature vector of the plurality of subjects, wherein the predictive model classifies the plurality of subjects as hydrated or dehydrated;
identifying at least one subject of the plurality of subjects that is classified as dehydrated;
generating candidate counterfactual feature vectors for the at least one subject, wherein the generating the candidate counter factual feature vectors comprises identifying counterfactual changes to the feature data elements which transition the predictive model classification of the subject from dehydrated to hydrated, where the candidate counter factual feature vectors comprise only identified counterfactual feature data elements;
computing a diversity distance between each possible pairing of candidate counter factual feature vectors;
generating a preference aware cost for the candidate counterfactual vectors;
generating an optimization score for each candidate counter factual feature vector using a weighted sum of the diversity score and the preference aware cost for each pair;
applying a greedy selection process to select a subset of the counterfactual feature vectors; and
providing the subset of the counterfactual feature vectors to the subject.

2. The method of claim 1, wherein a diversity distance comprises computing a first distance between each pairing of candidate counter factual feature vectors.

3. The method of claim 2, wherein the computing the first distance comprises calculating a first weighted L1 distance between each pair.

4. The method of claim 3, wherein the first weighted L1 distance is calculated using first weights, wherein the first weights comprise a diversity measure assigned to each feature type of the candidate counter factual feature vectors.

5. The method of claim 1, wherein the generating a preference aware cost comprises identifying a subset feature vector of the feature vector, wherein the subset feature vector includes feature data elements corresponding to counterfactual feature data elements of the candidate counter factual feature vectors.

6. The method of claim 5, wherein the generating a preference aware cost comprises computing a second distance between each pairing of candidate counter factual feature vectors and the subset feature vector.

7. The method of claim 6, wherein the second distance comprises a second weighted L1 distance between each pair.

8. The method of claim 7, wherein the second weighted L1 distance is calculated using second weights, wherein the second weights comprise preferences of the subject to adopt behavior to achieve counterfactual changes to the feature data elements.

9. The method of claim 1, wherein the generating candidate counterfactual feature vectors comprises applying a counterfactual generator to the counter factual feature vector of the at least one subject of the plurality of subjects.

10. The method of claim 1, wherein the feature data elements include fluid intake.

11. The method of claim 1, wherein the feature data elements include activity levels.

12. The method of claim 1, wherein the feature data elements include sweat loss.

13. The method of claim 1, wherein the feature data elements include ambient temperature.

14. The method of claim 1, wherein the feature data elements include relative humidity.

15. The method of claim 1, wherein the feature data elements include total body water.

16. The method of claim 1, wherein the feature data elements include time of day.

17. A system comprising,
one or more applications running on at least one processor of a remote server, the one or more applications communicatively coupled with a mobile device application running on a processor of a mobile device, the one or more applications configured to:
receive a feature vector for a plurality of subjects, wherein a feature vector comprises feature data elements of a subject;
apply a predictive model to the feature vector of the plurality of subjects, wherein the predictive model classifies the plurality of subjects as hydrated or dehydrated;
identify at least one subject of the plurality of subjects that is classified as dehydrated;
generate candidate counterfactual feature vectors for the at least one subject, wherein the generating the candidate counter factual feature vectors comprises identifying counterfactual changes to the feature data elements which transition the predictive model classification of the subject from dehydrated to hydrated, where the candidate counter factual feature vectors comprise only identified counterfactual feature data elements;
compute a diversity distance between each possible pairing of candidate counter factual feature vectors;
generate a preference aware cost for the candidate counterfactual vectors;
generate an optimization score for each candidate counter factual feature vector using a weighted sum of the diversity score and the preference aware cost for each pair; and
apply a greedy selection process to select a subset of the counterfactual feature vectors;
provide the subset of the counterfactual feature vectors to the one or more applications for display to a user.
